(19) United States
(12) Patent Application Publication
(10) Pub. No.: US 2007/0299306 A1
(43) Pub. Date: Dec. 27, 2007

(54) PROBE ASSEMBLY FOR ENDOSCOPIC PROCEDURES

(76) Inventors: Vinod K. Parasher, Rehobeth Beach, DE (US); Norman J. Miller, Georgetown, DE (US)

Correspondence Address: Connolly Bove Lodge & Hutz LLP, P.O. Box 2207, Wilmington, DE, 19899-2207, US

(21) Appl. No.: 11/764,252

(22) Filed: Jun. 18, 2007

Related U.S. Application Data
(60) Provisional application No. 60/815,512, filed on Jun. 21, 2006.

(51) Int. Cl. A61B 1/00

(52) U.S. Cl. 600/106; 600/104

(57) ABSTRACT

A probe assembly for endoscopic procedures includes a gear motor having a rotatable gear shaft through which a such as a wire or hollow needle is inserted with the probe being clamped to rotate by the gear motor. The probe extends through a catheter and the catheter and probe extend through a medical scope to the location to be viewed by the scope, such as a bile duct. Where the probe is a hollow needle, a stylet may extend outwardly of the needle and jointly rotate with the needle.

### CROSS-REFERENCE TO RELATED APPLICATION

This application is based on provisional application Ser. No. 60/815,512, filed Jun. 21, 2006, all of the details of which are incorporated herein by reference thereto.

### BACKGROUND OF THE INVENTION

Various endoscopic procedures are used which involve the insertion of probe type devices such as a wire or needle into difficult to reach portions of the body for various medical purposes. For example, Endoscopic Retrograde Chlongio Pancreato Graphy (ERCP) utilizes a wire inserted into the bile duct. Since the bile duct is a difficult to reach area, there are difficulties with the conventional prior art techniques in locating the bile duct entrance. For example, it is necessary for the wire to enter the bile duct opening (pappila of vater) which might be only a 5 millimeter opening in order to locate the endoscope viewing area in the bile duct and pancreatic duct. This is conventionally done under fluoroscopy and conventionally is a blind process where a thin wire inserted through a catheter blindly probes by twirling or rotating the wire until the opening is found. It would be desirable if some technique could be developed wherein the surgeon can have a more convenient and more accurate procedure for inserting the wire into the bile duct opening.

### SUMMARY OF THE INVENTION

An object of this invention is to provide improvements in the ERCP procedure which avoids the problems of the prior art.

A broad aspect of this invention involves providing a lumen finding steerable guide wire usable for various purposes.

In accordance with one aspect of this invention a probe assembly is provided wherein a wire or needle, such as a hollow needle, is clamped to a hand held drive mechanism in order to rotate the wire or needle so that the wire or needle can be readily moved to better locate the difficult to reach areas including the bile duct or pancreatic duct. Where a wire is used the wire could function as a guide wire to facilitate the insertion of the catheter and the endoscope viewing area into the difficult to reach location. The assembly would be used as a biopsy device for obtaining samples in difficult to reach locations.

The invention may also be practiced where the probe is a hollow needle, rather than a wire, which would be used for removing specimens, such as tumor fragments. A stylet would be inserted through the needle and would be mounted for joint rotation with the needle. The stylet could serve the multiple functions of performing as a drill for puncturing the intestine and for facilitating the larger diameter needle passing through the intestine wall. An additional function of the rotating stylet would be to penetrate the tumor to also facilitate the needle entering the tumor. A further function of the stylet would be to dislodge or breakup portions of the tumor. When the stylet is removed from the needle, the tumor fragments could more easily be aspirated through the needle to be collected for test purposes. In addition, the stylet would perform the normal stylet function of preventing clogging of the needle.

### BRIEF DESCRIPTION OF THE DRAWINGS

FIG. 1 schematically illustrates a probe assembly for endoscopic procedures in accordance with one aspect of this invention; and

FIG. 2 is an elevation view partly in section of a modified form of this invention.

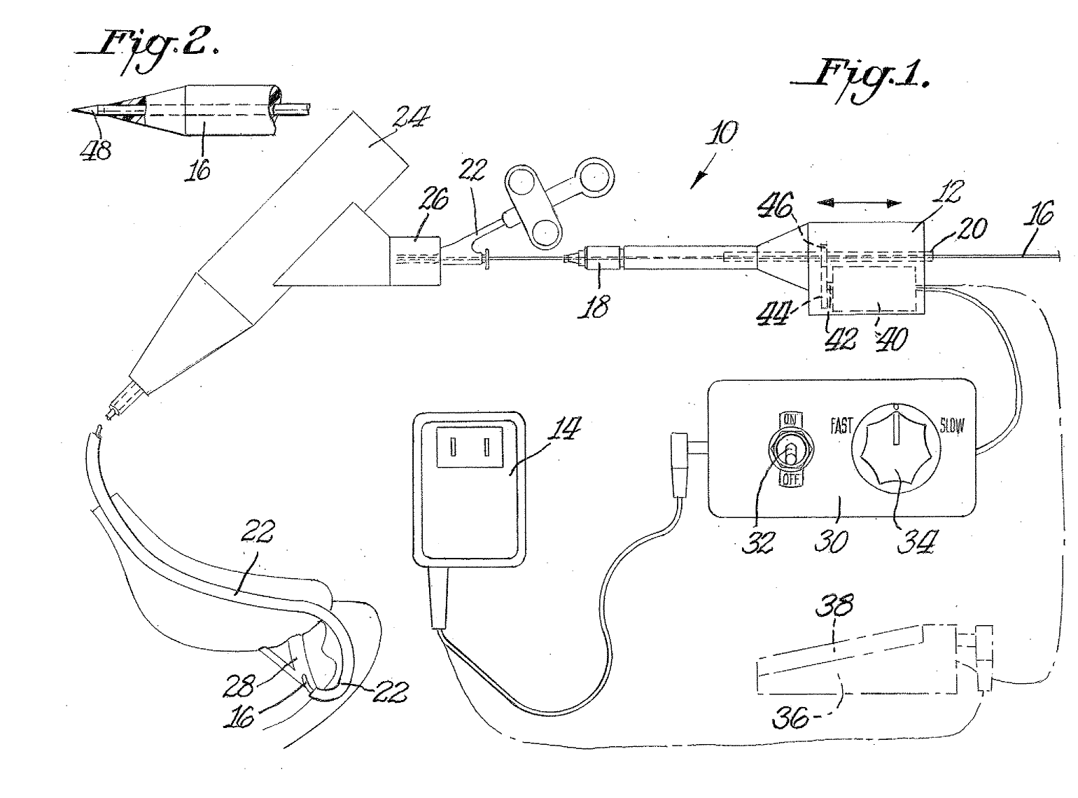

### DETAILED DESCRIPTION

The present invention, in general, involves providing a lumen finding steerable guide wire or a hollow specimen collecting needle which could be incorporated in a system as an elongated flexible probe attached to an endo-scope. The probe is clamped to a rotating mechanism which may be actuated in any manner such as by a foot pedal or hand operated mechanism to rotate the probe. The use, for example, of a foot pedal permits the surgeon to control the rotation and probe movements which could range from speeds of 15 rpm to 8,000 rpm or more, preferably 100 rpm to 3,000 rpm or 4,000 rpm, and most preferably 300-500 rpm.

The probe may be a wire which would act as a guide wire for finding an opening such as the bile duct opening to facilitate the movement of a catheter through that opening and into a difficult to reach location, such as a bile duct and/or pancreatic duct, for viewing by the endoscope. Alternatively, the probe could be a fine needle whereby the vibrating needle would break up cancer cells of a tumor to increase the yield of cells retrieved through aspiration or other conventional techniques.

The needle could be a hollow needle for collecting specimens, such as tumor fragments wherein the hollow needle is provided with a wire or stylet which extends outwardly from the needle. The stylet would rotate jointly with the needle and thus would facilitate the needle passing through, for example, an intestine wall and then into the tumor by the stylet first forming a smaller diameter hole in the manner of a drill wherein the larger diameter needle then enlarges the hole. The stylet would also function to fragment the tumor. After the stylet is removed from the needle, the fragmented tumor could more readily be aspirated through the hollow needle.

Although specific reference has been made to use of the invention in the bile duct and pancreatic duct, the invention could be used in any suitable body orifice, such as a duct, vein, artery, urethra and intestine.

Where the probe is a guide wire the wire would be of floppy or flexible structure so that the longitudinal movement of the wire readily finds its way into the opening which then permits more conventional procedures such as used in ERCP to be used. Such guide wire could be removed or left in place. While specific reference is made to the bile duct the same practices could be used for other difficult to locate regions such as the pancreatic duct.

The invention could be used to identify mass tumors in a manner more effective than MRI and to facilitate performing ERCP and its associated uses.

Where the probe is a fine needle the needle would pass through areas such as the pancreas and would be used to break cancer cells which would then be removed through suction or aspiration such as by a suction syringe where the materials are squirted onto a slide. Sufficient passes could be used to obtain the desired amount of test tissue. The needle could be the pointed tip of the probe which would be inserted into a tumor to break the cells as a result of the longitudinal movement of the needle thereby facilitating suction retrieval. If desired, the needle may have an axial stylet to facilitate unblocking the needle as the needle moves.

Preferably, the probe is driven by a high variable speed mechanism which may be controlled by a foot pedal. It is to be understood, however, that other actuating and speed control structures could be used within the practice of the invention. The probe is preferably clamped with a wire clamp collet to permit quick load and release. This subassembly permits the surgeon to longitudinally move the probe, so that the probe can be extended or retracted within a catheter as desired without affecting the rotation of the probe. These features create great potential for a successful process. This subassembly is then preferably attached to an endoscope so that the distal end or tip of the probe can be viewed.

The single FIGURE schematically illustrates a probe assembly 10 for endoscopic procedures. As shown therein the assembly is powered with a D.C. gear motor 12 utilizing an AC/DC adapter 14. The speed of motor 12 is variable permitting desired preselected speeds to be used. A wire 16 which would be a catheter wire of extended, in effect, indefinite length is gripped by a slide collet 18 which can be readily locked and released.

The “infinite” length wire 16 is threaded through the back end of the hollow gear shaft 20 in gear motor 12 through the collet clamp 18 into a catheter 22 of known conventional construction. The wire 16 extends to the far end of the catheter 22 at which time after clamping the wire 16 with the collet clamp 18 the motor 12 is energized rotating the wire 16 at the desired speed.

If needed the clamp 18 could be released and additional wire could be advanced through the catheter 22.

The small hand-held motor assembly can be extended or retracted while the wire is clamped and running or rotated which provides the surgeon or physician the necessary control the perform the procedure.

From catheter 22 the wire extends into a medical scope or endoscope 24 which could also be of known conventional construction.

As illustrated in the drawing the wire 16 extends into the endoscope 24 by first passing through a passageway in block 26. The subassembly of gear motor 12 and catheter 22 could be secured to a main body or support not shown. If desired endoscope 24 could be detachably mounted by any suitable clamp mechanism to the main body or support or to any convenient portion of the subassembly comprising gear motor 12 and catheter 22. Such clamp assembly could, for example, be a pair of pivotally mounted clamp members each of which has an arcuate groove or indent for receiving the circular endoscope body. Catheter 22 extends through scope 24 in block 26 with the wire 16 extending out of catheter 22. Catheter 22 and wire 16 are inserted into the area near, for example, the bile duct 28. The wire 16 can then be moved back and forth by moving the gear motor 12 as shown by the double arrowhead 30 until the wire 16 enters the bile duct opening at which point the endoscope can view the bile duct and at that point normal ERCP procedures can be used.

The speed of rotation of wire 16 can be controlled by any suitable speed control device. For example, the drawing illustrates a known type of electrically operated speed control mechanism 30 having an on/off switch 32 and a hand operated rotatable speed control selector 34. Speed control mechanism 30 is shown to be electrically connected to the gear motor 12.

Alternatively, the rotational speed of wire 16 could be controlled by a foot pedal speed control 36 having a variable speed control selection which is actuated by pivoted actuating surface 38. Variable speed foot pedal 36 is shown to be electrically connected to the gear motor 12. More particularly, as shown in the drawing the gear motor 12 includes a variable speed motor 40 having a drive shaft 42 which drives a gear 44 connected through a gear train to or connected directly to a gear 46 on the shaft 20 through which wire 16 extends so that rotation of the shaft 20 causes wire 16 to also rotate.

Because wire 16 enters the gear motor 12 through an unobstructed back portion, any length of wire could be used. Accordingly, the length of wire is referred to herein as being of infinite length.

Among the advantages of the assembly 12 are that the assembly is powered electrically. Accordingly, there is no need for compressed air to power the assembly. In addition, the wire length is not limited by any constraints composed by the assembly. Further, in the option of having a foot pedal 36 the physician has the capability to control the speed at will.

The present invention could be utilized to provide a lumen finding steerable guide wire. For example, in prior practices consistent deep intubation of the small bowel remains problematic despite the availability of various methods. Current methods require fluoroscopy or long endoscopes. The present method not only will allow safe placement of guide wires consistently beyond the ligament of trietz or deeper during endoscopy without fluoroscopy, but also is convenient and is not time consuming. Such method may assist in the placement of feeding tubes, modified wireless capsule endoscopes, double balloon endoscopes and/or standard endoscopes.

The present invention has been used to evaluate the placement of a steerable lumen finding guide wire in an ex vivo sample of porcine small bowel. In a practice of this invention a mechanical device allows 360 degree rotation of a flexible guide wire at varying speeds. Simultaneous incremental feeding of the rotating guide wire is also possible. A 460 cm. 0.36 inch guide wire (Jagwire, Boston Scientific, Nettick, Mass.) was attached to the device. A similar length of porcine small bowel was placed and fixed in a water bath for experimentation. In the results of this practice the small bowel was transversed by the guide wire in its entirety by this method. Continuous deflection of the rotating guide wire tip against the small bowel mucosa coupled with incremental feeding allowed the lumen finding and, thus, the deep entral access. This method thus allows deep entral access by a mechanically steerable rotating lumen finding guide wire.

Among the advantages of the present invention are the following:
1. Device is able to rotate and pass long guidewire (260-460 cm);
2. Will allow access to the entire length of small and large bowel;
3. Will allow diagnosis and treatment of small and large bowel disorders;
4. Will allow placement of feeding tubes, modified wireless devices, enteroscopes, and double balloon endoscopes; and
5. Diagnosis and treatment of stricture of body cavities and duct, e.g. bile, pancreas, urinary system, heart.

It is known to provide hollow needles with thin wires or stylets to prevent the needle from clogging. In conventional practices the stylet could be moved back and forth axially to unclog such hollow needle. FIG. 2 illustrates a departure from conventional practices. As shown therein, the probe 16 is in the form of a hollow needle having a stylet 48. Stylet 48 is preferably snugly received in the hollow needle along the longitudinal axis of the needle. By virtue of the tight fit in the needle and/or by co-mounting stylet 48 with the rotating mechanism, when the needle 16 is rotated (as previously described) the stylet 48 rotates along with the needle.

In accordance with this invention stylet 48 extends outwardly from needle 16 any suitable amount such as 1 millimeter. Because stylet 48 extends outwardly of needle 16 and because stylet 48 is of smaller diameter than needle 16 and finally because stylet 48 rotates along with needle 16 a number of advantages or benefits result. The rotating stylet and needle function in the manner of a drill which would puncture the intestine wall, for example, and then any hard tumor. The smaller diameter stylet by extending outwardly from needle 16 is able to penetrate the intestine wall more readily than the larger diameter needle. When the needle itself reaches the intestine wall a hole has already been formed by the stylet 48. It is therefore easier for the needle 16 to pass through and enlarge the hole in the intestine wall. Similarly, when the stylet 48 reaches a hard tumor it is easier for the smaller diameter stylet to puncture the tumor and correspondingly it is easier for the larger diameter needle to follow into the hole created by the stylet and enter the tumor. A further advantage of the rotating stylet is that the stylet would break up or fragment the tumor. After there has been sufficient fragmentation the stylet 48 would be completely removed from needle 16. A suitable aspiration device would be mounted to the upstream end of needle 16 and the fragmented tumor would more readily pass through the hollow needle 16. The provision of a stylet extending outwardly from the hollow needle and rapidly rotating along with the hollow needle thereby facilitates use of the device in puncturing the intestine wall and otherwise entering the abdominal cavity or any other suitable site.

The invention has been particularly described with respect to a rotating wire or needle that can be moved longitudinally. Various concepts of the invention can also be practiced wherein the assembly itself provides the capability of vibrating the wire longitudinally in the manner described in U.S. Pat. No. 7,048,684, all of the details of which are incorporated herein by reference thereto.

What is claimed is:

1. A probe assembly for endoscopic procedures comprising a gear motor having a rotatable gear shaft through which a probe may be inserted whereby the probe may be rotated upon rotation of the gear shaft, a clamp mechanism for clamping the probe, a catheter downstream from the clamp mechanism whereby the probe may extend out of the clamp mechanism and into the catheter, and an endoscope which may be mounted to the catheter whereby the catheter and probe may extend through the endoscope to be disposed at a site of view from the endoscope.

2. The assembly of claim 1 wherein the clamp mechanism is a collet.

3. The assembly of claim 1 wherein the probe is a wire.

4. The assembly of claim 1 including a variable speed unit for controlling the speed of rotation of the probe.

5. The assembly of claim 4 wherein the variable speed unit is a foot pedal speed control.

6. The assembly of claim 4 wherein the variable speed unit includes an off/on switch and a manually operable speed selection dial.

7. The assembly of claim 1 wherein the probe is a hollow needle, and a stylet mounted longitudinally within and extending out of said needle.

8. A probe assembly for endoscopic procedures comprising a gear motor having a rotatable gear shaft through which a probe may be inserted whereby said probe may be rotated upon rotation of said gear shaft, a clamp mechanism for clamping said probe, an endoscope mounted downstream from said clamp mechanism, said probe extending through said endoscope, said probe being a hollow needle having a stylet longitudinally mounted within said needle and extending outwardly of said needle, and said stylet being mounted for joint rotation with said needle.

9. The assembly of claim 8 wherein said stylet is removable from said needle whereby an aspiration device may be mounted to an upstream end of said needle to aspirate fragments through said hollow needle.

10. In a method of endoscopic retrograde chlongio pancreato graphy and the like including the steps of inserting a probe into the drive shaft of a gear motor, clamping the probe to the gear motor so that the probe is rotated by the gear motor, inserting the probe into a catheter, extending the catheter and the probe through a medical scope until the catheter and probe are located at an intended body orifice, rotating the probe while the probe is at the intended body orifice, longitudinally moving the probe by movement of the gear motor, and viewing the probe at the intended body orifice.

11. The method of claim 10 wherein the probe is a wire which extends unimpeded through the back end of the gear motor.

12. The method of claim 11 including selecting the speed of rotation of the probe by a variable speed device.

13. The method of claim 12 wherein the selection of speed is achieved by stepping on a foot pedal speed control.

14. The method of claim 10 wherein the intended body orifice is the bile duct.

15. The method of claim 10 wherein the intended body orifice is the pancreatic duct.

16. The method of claim 10 wherein the probe is a hollow needle, inserting a stylet longitudinally into the needle with the tip of the stylet extending outwardly from the needle, and jointly rotating the stylet and needle.

17. The method of claim 16 including puncturing an intestine wall by the stylet first puncturing the wall and then by the needle penetrating the hole created by the stylet to enlarge the hole and permit the needle to pass through the wall.

18. The method of claim 17 including puncturing a tumor beyond the wall by the stylet first puncturing the tumor to initially create a hole and by the needle then entering the hole and enlarging the hole, and fragmenting the tumor by the rotating action of the stylet.

19. The method of claim 18 including removing the stylet from the needle, applying an aspiration device to an upstream end of the needle, and aspirating tumor fragments through the needle.

20. The method of claim 16 including removing the stylet from the needle, applying an aspiration device to an upstream end of the needle, and aspirating tumor fragments through the needle.
